(19) United States
(12) Patent Application Publication
(10) Pub. No.: US 2025/0185947 A1
(43) Pub. Date: Jun. 12, 2025

(54) Use of patient vital sign data for preventing medical errors

(71) Applicants: Jeremy Straub, Fargo, ND (US); Jared Maul, West Fargo, ND (US)

(72) Inventors: Jeremy Straub, Fargo, ND (US); Jared Maul, West Fargo, ND (US)

(21) Appl. No.: 18/963,079

(22) Filed: Nov. 27, 2024

Related U.S. Application Data
(60) Provisional application No. 63/605,330, filed on Dec. 1, 2023.

(51) Int. Cl. A61B 5/117; G16H 40/20

(52) U.S. Cl. CPC A61B 5/117; G16H 40/20

(57) ABSTRACT

Patient misidentification is a preventable issue that contributes to medical errors. When patients are confused with each other, they can be given the wrong medication or unneeded surgeries. Unconscious, juvenile, and mentally impaired patients represent particular areas of concern, due to their potential inability to confirm their identity or the possibility that they may inadvertently respond to an incorrect patient name (in the case of juveniles and the mentally impaired). This disclosure evaluates the use of patient vital sign data, within an enabling artificial intelligence (AI) framework, for the purposes of patient identification. The AI technique utilized is both explainable (meaning that its decision-making process is human understandable) and defensible (meaning that its decision-making pathways cannot be altered, just optimized). It is used to identify patients based on standard vital sign data. Analysis is presented on the efficacy of doing this, for the purposes of catching misidentification and preventing error.

### PRIORITY

This application claims the benefit of U.S. Provisional Application Ser. No. 63/605,330, filed Dec. 1, 2023, which application is incorporated herein by reference in its entirety.

### SUPPORT STATEMENT

This invention was made with government support under grant #P20GM103442 awarded by the National Institute of General Medical Sciences of the U.S. National Institutes of Health. The government has certain rights in the invention.

### BACKGROUND

Medical mistakes are a significant problem. They range from systematic failures to isolated accidents and provider issues. A study, in 2013, estimated that medical errors cost over USD 20 billion each year and result in the death of 100,000 people.

Patient misidentification is a component of this issue and can result when providers lose track of which locations patients are assigned to, swap charts, or otherwise confuse one patient with another. Notably, a 2016 study found that the problem actually starts in patient registration, with patient misidentification at registration being the leading cause of misidentification, generally. Each hospital, on average, loses USD 17.4 million per year due to misidentification-attributable denied claims, and 86% of providers “have witnessed or have known of” a misidentification-attributable medical error.

Patient misidentification has been labeled a “wicked” problem. It drains staff time, costing “clinicians” approximately 30 min during each shift searching for records, and costs hospitals millions of dollars in lost revenue. If patients are given the wrong procedure or medication, it can result in injury or even patient death. A study at the Veterans Health Administration, for example, found that, in 31% of reported misidentification incidents, a procedure was performed on the wrong patient. In other cases, misidentification can result in a failure to diagnose a condition or inform the patient of the diagnosis, delaying treatment. The impact of misidentification can even extend beyond the death of the patient, as it can impair the process of relatives claiming or moving a deceased person's corpse.

There are potential legal risks to providers and facilities from misidentification, which include liability under the torts of battery, false imprisonment, and emotional distress. A study found that wristbands were missing in 70% of cases, nearly 8% of cases had illegible wristbands, and over 20% of cases had missing, erroneous, or conflicting ID information. In only 1% of cases, though, the wristband was actually on the wrong patient.

Another study found that hurried staff were partially responsible for 64% of misidentifications and that an identification policy was not followed in nearly 50% of incidents. Language issues (46%), missing ID bands (38%), patients answering to incorrect names (38%), staff carelessness (35%) and using yes/no questions for identification (33%) were also large contributors.

A study of laboratory medicine identified patient identification as being responsible for “182 of 253 adverse events” and identified causes including admissions identification issues, mislabeling specimens, not using two sources of patient identification, and not using two-person verification as being responsible. These incidents resulted in issues with cancer diagnoses, blood transfusions to incorrect patients (including incidents which resulted in incompatible blood being transfused) and information being placed in incorrect patient records.

A study of neonatal intensive care units found significant potential for misinformation. They found that approximately 50% of patients were at risk for potential misidentification due to similarities with their medical record numbers, name similarities, and common surnames.

A study of registration issues found that they occurred between seven and fifteen times each month due to issues with IT systems, training issues and not having a single index of patients. Another study examined the impact of computerized physician order entry on medical errors, noting that orders on misidentified patients occurred just under 1% of the time (across all orders) and had correlations with provider fatigue factors. Other studies found name similarity issues and missing wrist bands to be leading causes of misidentification.

### BRIEF DESCRIPTION OF THE DRAWINGS

In the drawings, which are not necessarily drawn to scale, like numerals may describe similar components in different views. Like numerals that have different letter suffixes may represent different instances of similar components. Some embodiments are illustrated by way of example, and not limitation, in the figures of the accompanying drawings.

FIG. 1 is a diagram of a gradient descent expert system training algorithm, according to an embodiment.

FIG. 2 is a diagram of a patient misidentification prevention system, according to an embodiment.

FIG. 3 is a diagram of a first GDES network, according to an embodiment.

FIG. 4 is a diagram of a second GDES network, according to an embodiment.

FIG. 5 is a diagram of a third GDES network, according to an embodiment.

### DETAILED DESCRIPTION

Systems and methods described herein use of an artificial intelligence-based solution, which utilizes patient vital sign data, for the purposes of patient identification. It assesses whether, using this approach, patients can be readily identified to a level that would be useful for preventing misidentification. It also characterizes the level of misidentification prevention accuracy that is possible using the proposed approach.

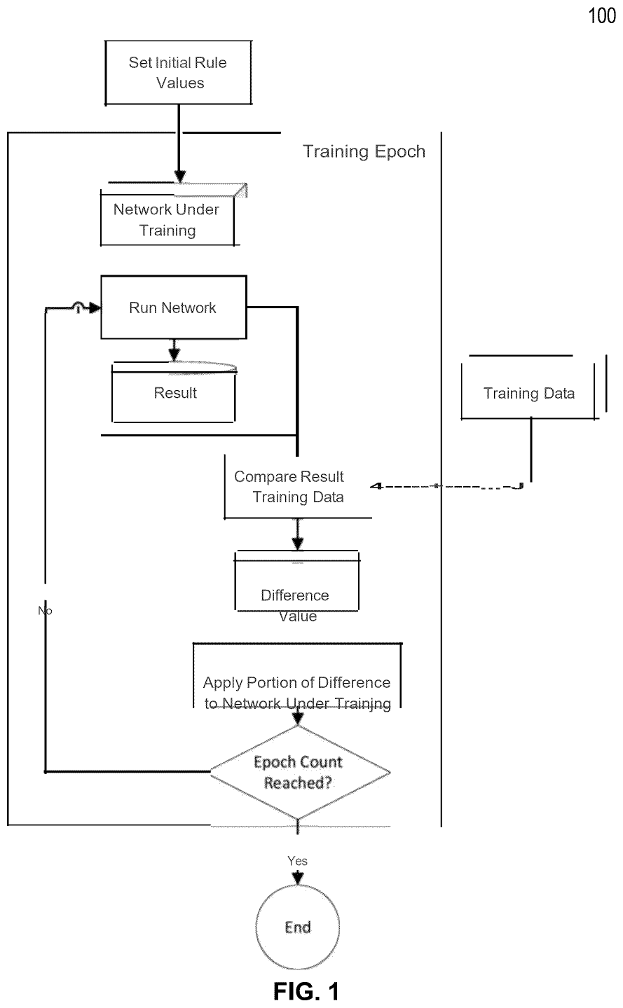

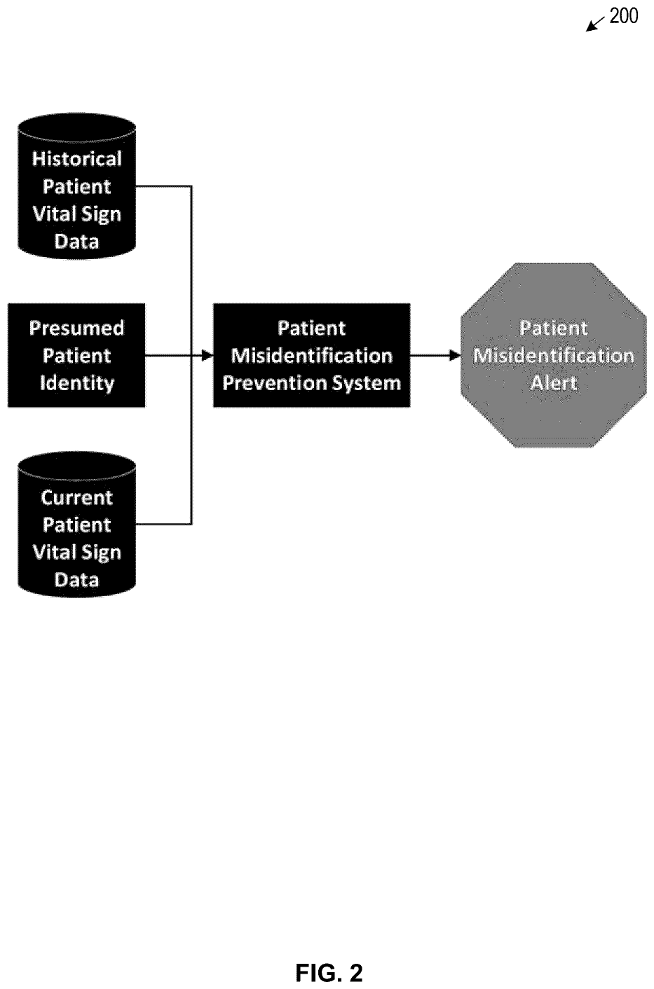

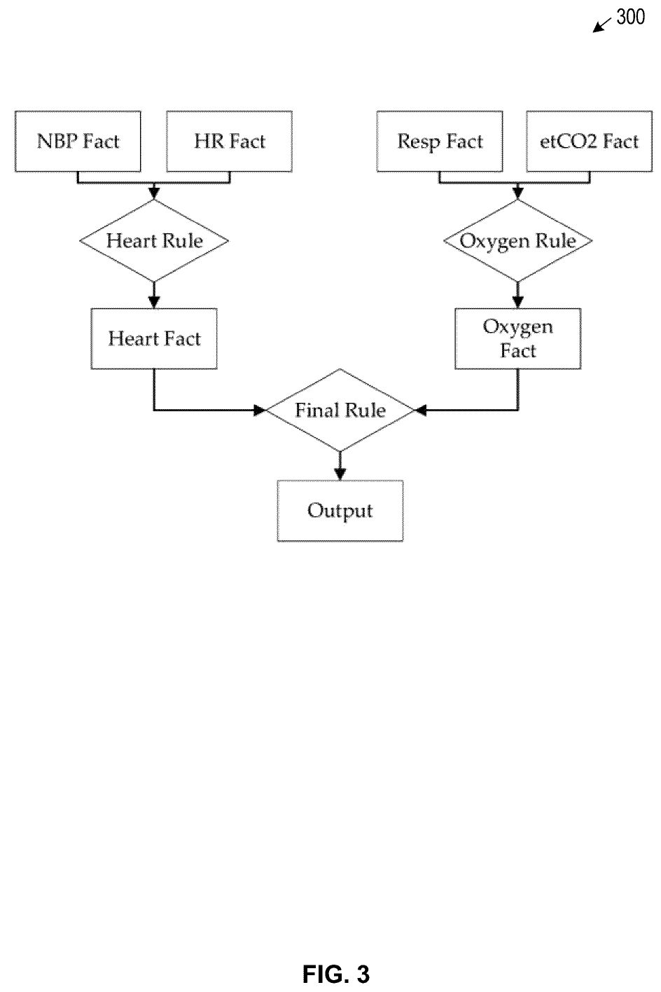

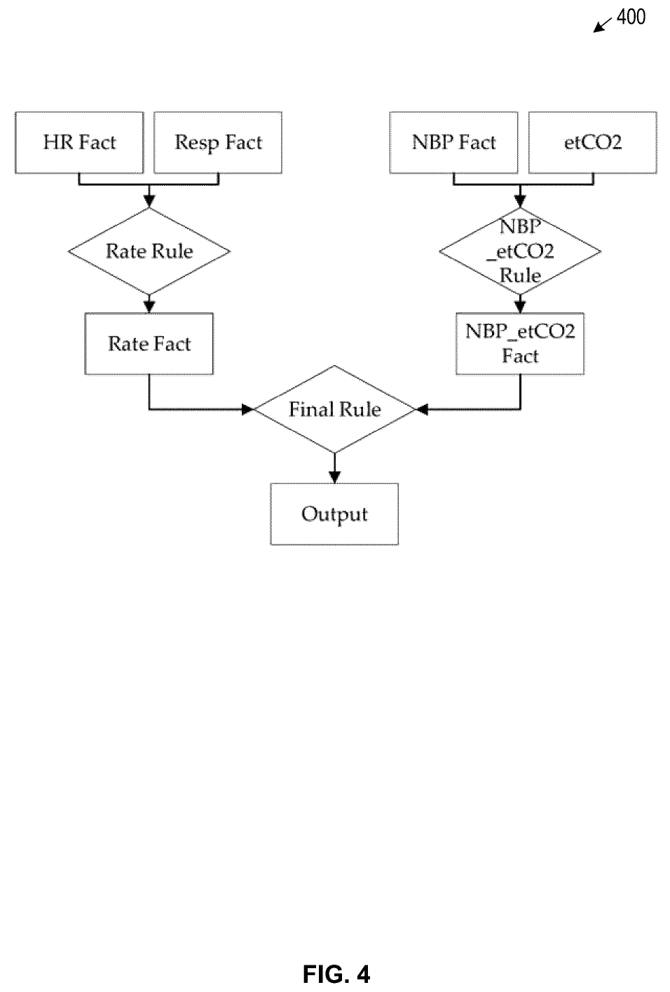

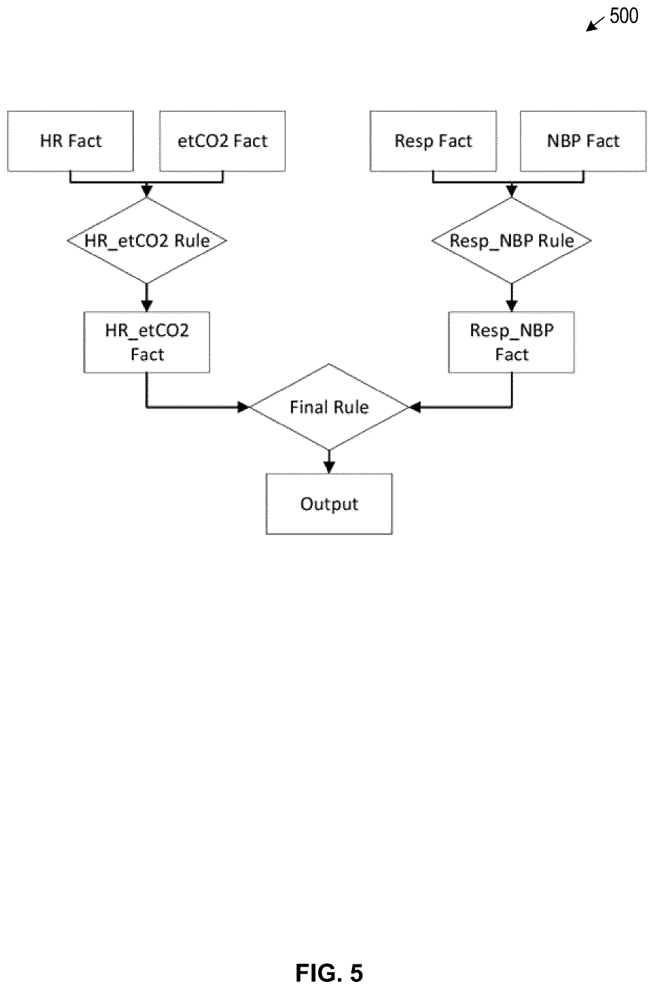

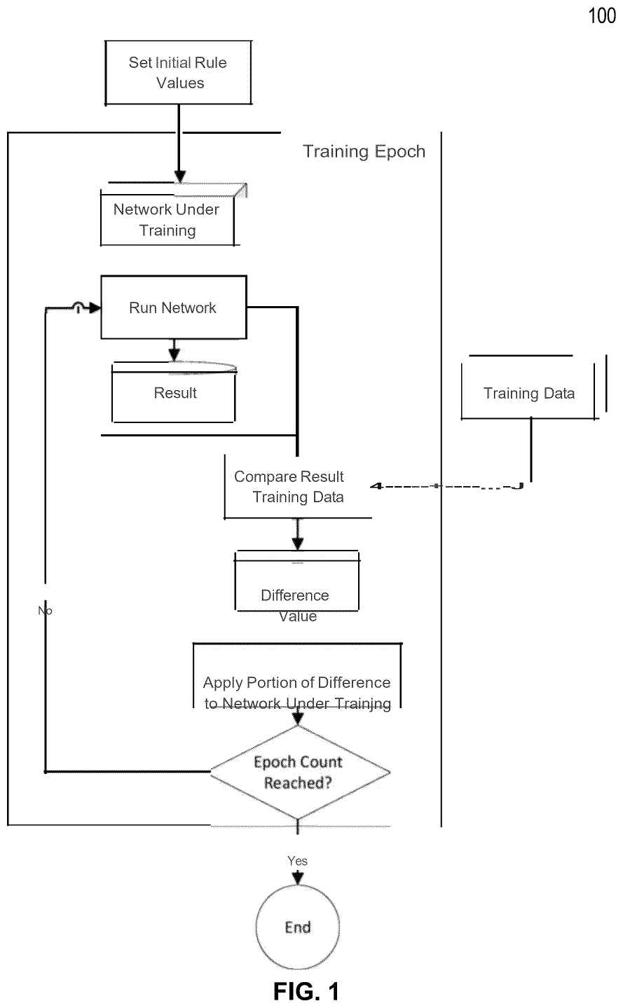

FIG. 1 is a diagram of a gradient descent expert system training algorithm 100, according to an embodiment. The gradient descent expert system training algorithm 100 shows a gradient descent-style backpropagation learning algorithm on the expert system's rule-fact network.

This system uses a gradient descent-style backpropagation learning algorithm on the expert system's rule-fact network. The rule-fact network is constructed by a human system developer, based on their understanding of the phenomena being modeled, and can be refined through performance testing, as needed. The backpropagation algorithm changes the weightings of rules' inputs to better match the phenomena and reduce error, during the training process. To do this, a fraction of the error (between the current system output and target value for a training run) is proportionally allocated to each rule, based on its contribution to the output value. The algorithm for system training is depicted in FIG. 1.

These AI systems are able to improve their performance through unsupervised, semi-supervised, and supervised learning processes. One of the most common forms of supervised learning is gradient descent, where corrections are applied iteratively to produce outputs closer to a goal. Backpropagation, which alters a network's weightings working from the end of the network toward the beginning, is one type of gradient descent. Expert systems are another AI technique, which are loaded with decision pathways from a human expert and are not typically used with machine learning.

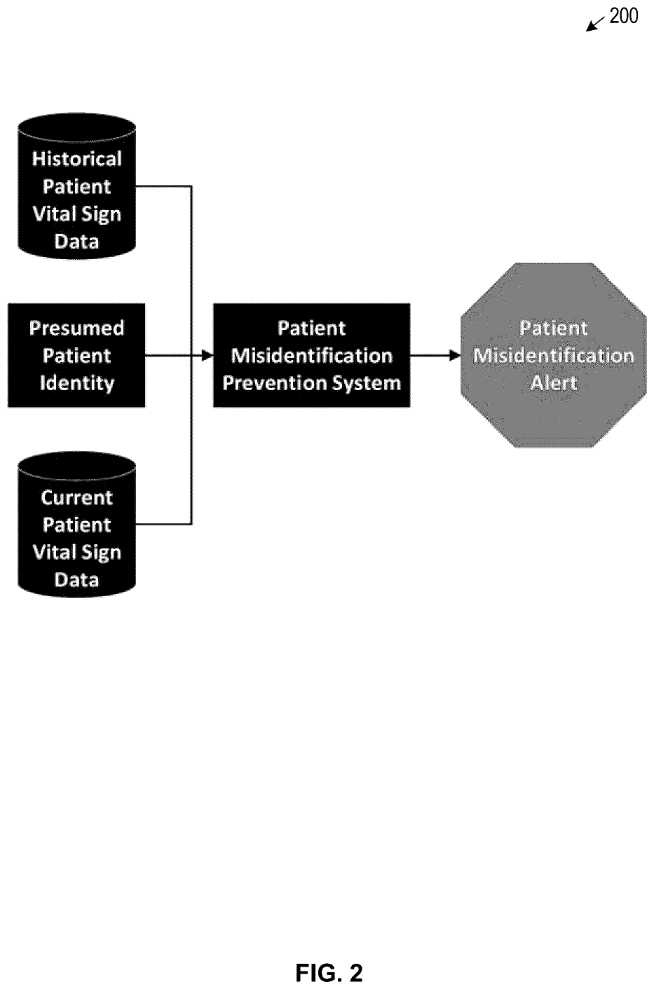

FIG. 2 is a diagram of a patient misidentification prevention system 200, according to an embodiment. To effectively use vital sign data for patient misidentification prevention, the vital sign data must be available to be correlated with patient identity information. Two scenarios for use are illustrative of how this could be achieved (though others are also possible).

The first scenario is a smart hospital bed which would contain or be connected to the vital sign monitoring equipment and know the supposed identity of the patient occupying it.

Under this scenario, if the bed were to detect an anomaly it could notify hospital staff, display an alert, and annotate any patient identity information with a misidentification concern. Notably, in addition to detecting possible patient misidentification, the same techniques proposed herein could be useful for detecting changes in medical condition; thus, any change would need to be investigated to confirm both patient identity and condition.

The second scenario is a patient record-keeping system. This system would be conceptually similar to the smart bed; however, it would not require specialized bed hardware. Instead, the system would look for indications that new incorrect information is being loaded for a patient, through the loading of vital signs data associated with the patient. If this data indicated a potential problem, patient misidentification would be suspected, and other contemporaneously loaded information would be flagged with the concern. Additionally, any access to the record for performing a procedure or dispensing a medication would trigger a misidentification concern alert to the provider.

Under both scenarios, the system would start with a presumed patient identity and historical vital sign data for the patient. It would collect or have vital sign data provided to it. The historical and current vital sign data, along with the presumed identity, would be provided to the misidentification system. This system would assess the likelihood of misidentification, using the process described subsequently and, if needed, provides a patient misidentification alert.

If no prior data are available for the patient, the system would not be able to provide misidentification warnings until data has been collected for a period of time. Thus, when prior data are unavailable, or where it has been invalidated by a change in a patient's medical condition (i.e., where an alert has been generated, the patient identity reverified and the system instructed that the identification is correct), the system would enter an initial data collection mode. While in this mode, it could caution users that it is learning the patient and not yet able to provide misidentification alerts, thus encouraging providers to be particularly careful. This could be augmented with provider practices that include additional verification, while the system is in this mode, to ensure that the patient is correctly verified manually (and, thus, also assuring that the data that is being collected will be associated with the correct individual).

A key decision, for system implementation, is what data to analyze. Ideally, the system would operate using vital signs which are easily and already commonly recorded. This would facilitate the use of sensors that would already be needed for other purposes also being used for this application. This would reduce costs and collection burden on the patient. From the variety of vital sign information available, the following four pieces of information were selected for use: heart rate, end-tidal carbon dioxide, respiratory rate, and blood pressure. These four vital signs were selected primarily because of availability. Each patient within the dataset has an abundance of data for each of these vital signs and these data were available for all patients. A key area of future work will be to identify whether other vital signs perform as well as, outperform, or underperform the ones analyzed in herein. In addition to assessing performance generally, the correlation of vital sign performance with demographic characteristics also merits assessment in future work.

Each piece of vital sign information's numeric value is converted into a value between 0 and 1, to supply to the system for training. The data set contains a set of patients with vital sign data collected during surgery. Ten of these patients are used in this experiment. Each of these ten patients was selected because their procedures lasted longer than 70 min. Data used for training the system for a given patient were taken from times 30:00.00 through 39:59.99 of the patient's procedure. This ten-minute period allows for 60,000 entries for each of the four vital signs.

The system was tested using a series of trials. Trial numbers are common across the datasets that were used for analysis and refer to a specific set of steps that were used for data processing and analysis, in all instances.

For example, trial 1 is performed by supplying the network with training data from patient one to produce a single output value. This output value also lies between 0 and 1. Following this, a data sample is taken from each of the ten patients at intervals within times 1:00:00.00 and 1:09:59.99. These samples are supplied to the network and their output values are compared with the original value generated by training the network at the beginning of the trial. For trial 1, this means that patient one will have ten minutes of data used to train the network and produce an output value. Following this each of the ten patients will have a small amount of data supplied to the network, producing ten output values, each corresponding to one of the ten patients. Ideally, the value generated by the sample from patient one will be most similar to the original value produced by training the network. Meanwhile, the other nine patients should produce output that differs from the original value. For trials 1 through 10, patients one through ten are each used to train the network for their respective trials. This convention is followed for trials 11 through 50, with each collection of ten trials being modified slightly as described below. Each of these 50 trials is performed three times, to test three different networks. Each of these three networks is run four times to explore four slightly different approaches, which is also described below.

For the experimentation presented herein, converting the raw data to the value between 0 and 1 was completed using methods that vary by trial. In trials 1 through 10 and 21 through 50, the method used was to take the value and divide it by the maximum value found in any of the patients. For example, at 30:00.00 patient one has a heart rate of 53 bpm. This was divided by the maximum heart rate of all patients, in this case 135 bpm, resulting in a value of 000.393. This approach is referred to as the comprehensive data conversion method. Trials 11 through 20 divide the particular value by the greatest value found in a given patient's own data. This is referred to as the isolated data conversion method.

Both approaches could be used by a system operating in the real world and would simply use the largest value recorded to date; however, this would potentially necessitate caping values at 1.000, should a higher value be detected during operations, or retraining the network. In any case, for a real-world implementation, the divisor value would need to be set, potentially based on an in-situ pilot study building upon the experimentation, which is presented herein, to be consistent throughout operations. Notably, the largest value of all patients also approximates using the largest reasonable value, which would not change over time.

Once the values are converted, they are supplied to the system. Procedurally, this is completed using a set fact (SF) command (see). A 32-character globally unique identifier (GUID) is assigned to each fact and rule which is used for identifying nodes within the network when issuing commands. For example, the command below sets the heart rate input fact to 000.393:

$$SF : \{ 24 da 3290 - e 934 - 4 a 9 c - 84 e9 - 6 a 0 d 856 e 5073 \} = 0.393$$

Set fact commands are issued for each of the variables used in the given tests. Once the SF commands are issued, a training (TR) command is then issued. Training commands begin with a reference to a starting fact. The blood pressure fact is used as the starting fact and the second GUID included in the TR command is that of the output fact.

Note that one of the SF commands is vestigial and not strictly necessary, as the initial fact included in a TR command is set by the TR command; however, all SF commands were issued for simplicity, as this approach allows the TR command issued to be changed without requiring changing the block of SF commands (and the issuance of an additional SF does not materially impact system operations). An extended discussion of this can be found in Appendix A along with technical details regarding the system commands.

The four input values (and the starting fact value from the TR command) are drawn from the data row currently being used for training. For the output value, 000.500, which is the midpoint of the valid output range, is used in all cases. This approach makes the trained patient the middle output value with other patients able to show a positive or negative deviation from this patient.

The command set (which is presented in Appendix A) of four SF commands followed by one TR command, is repeated for each row of data in the patient spreadsheets (60,000 records). Following these commands, a present (PR) command is issued.

Two forms of the PR command were utilized. For some tests, a set value (e.g., 000.500) was used that is at or near the middle of the allowable fact value range. The value of 000.500 was used in trials 1 through 20 of each set of data. In trials 21 through 30 the value of 000.600 was used, trials 31 through 40 used 000.700, and trials 41 through 50 used 000.550. A second approach utilized, as input for the PR command, the blood pressure value from the last training row (to produce a natural output from the network). The form of this command was the same, except that the value assigned to blood pressure varied depending on the value in the final row.

A third approach was tested, which uses a fixed value (just like the first approach). The difference is that, just prior to the PR command being issued, all four facts are also set to that value.

Finally, a fourth and final approach is similar to the third, with one primary difference. The average value of each fact is calculated. These averages are used in a set of SF command and the PR command.

The results from these four different approaches to generating a baseline value for a given patient are compared herein. The output of the PR command is what all other results, which are based on performing PRs using data from later in the patient data sets, were compared against. This is referred to as the initial training output value or the target value.

It is important to note that this system is intended for use as an additional layer of protection against patient misidentification. It is not designed to uniquely identify patients, nor is it designed to guarantee that patients will be identified correctly in all circumstances. Rather, it is designed to augment existing patient identification and misidentification prevention methods. It will provide partial support or refutation for the presumed patient identity provided. This analysis can occur in tandem with other commonly used patient identification methods, such as verifying identity details with the patient and, in this context, it is capable of reducing patient misidentification. Further expanding the identity assessment capabilities of the proposed system with the use of additional data elements and methods is a potential topic for future work.

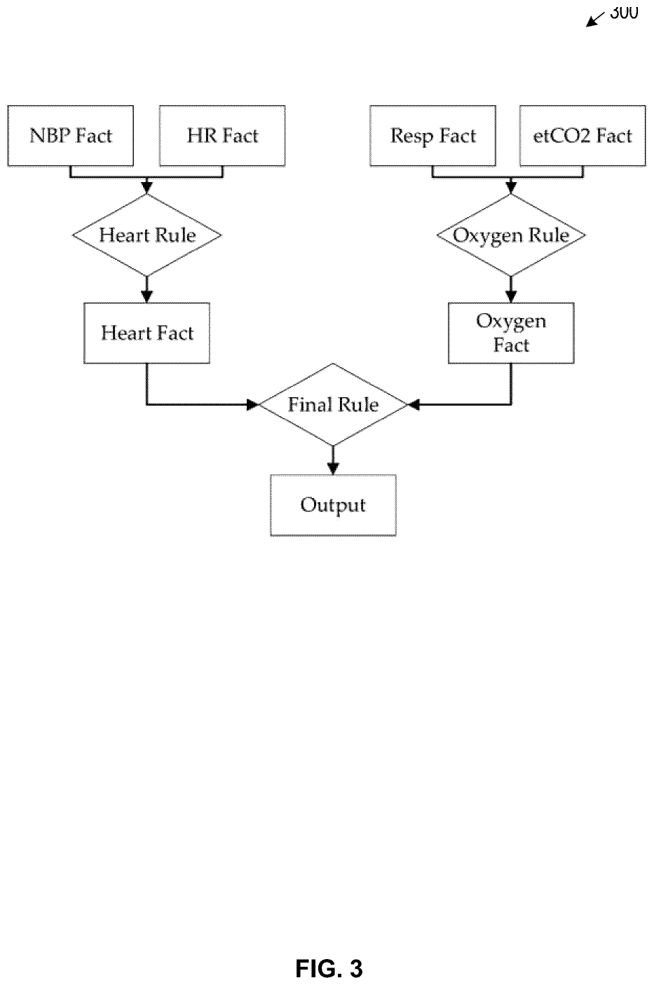

FIG. 3 is a diagram of a first GDES network 300, according to an embodiment.

The first network is comprised of seven facts and three rules. It uses a heart rule, which gets its value by combining blood pressure and heart rate data, as well as an oxygen rule, determined by combining respiratory rate and end-tidal carbon dioxide data. These two rules provide the heart fact and oxygen fact, respectively, which are inputs to the final rule and output fact. This model is presented in FIG. 3.

The data used to test this system was sourced from patient monitoring and vital sign data that was recorded during surgical cases where patients underwent anesthesia at the Royal Adelaide Hospital (RAH). The data are typical of biometric information that is frequently collected during hospital stays.

Data were utilized from 10 patients: case01, case03, case04, case05, case06, case09, case11, case12, case13, and case14 in the RAH dataset. These files correspond to patients one through ten, respectively, in the data presented in this disclosure (e.g., patient one is associated with case01, patient two is associated with case03). Patients whose procedures were completed in less than 70 min (e.g., case02, case07, case08, and case10), and thus did not have a full set of data, were excluded. Training data were sourced from the fourth file for each patient (e.g., uq_vsd_case01_fulldata_04.csv for patient 1), which contains data collected from the times 30:00.00 to 39:59.99. For each patient, 60,000 rows of data are included covering this timeframe (however, in many cases, values are repeated so that only a handful of unique values are present).

After the system was trained, data from later in patients' surgery was used to test it. The results were generated by presenting data from the seventh file of each patient (e.g., uq_vsd_case01_fulldata_07.csv for patient 1), which covers the time from 1:00:00.00 to 1:09:59.99. For each patient, seven rows were presented for data collection: these were rows 3 (rows 1 and 2 contain header data), 10,000, 20,000, 30,000, 40,000, 50,000, and 60,000. Each of these rows was used to create a group of set fact commands (in the same way as was used for the training data) and a PR command was then run. After each run, all intermediate facts were reset (using the SF command) to the default value of 000.500 before performing the next run. Data from this experimental process, using three different network configuration designs, are presented below.

The following table represents the results obtained using the first GDES network model, hence the header including Set 1. Trials 1 through 10 of Set 1 are presented in Table 1 and the first PR approach is used. The average error column displays, for each trial, how much all patients tend to deviate from the initial output training fact on average. The avg err column focuses on this deviation in just the target patient, rather than the average of all patients. The lowest column displays a yes or no value, depending on whether the target patient had the lowest deviation from the initial training output of all patients. The correct at columns also have a yes or no value representing whether or not the target patient falls within a given margin of error.

The false at column represents how many of the incorrect patients also fall within that margin of error. An example of how to read row one is as follows: In set 1, trial 1, the average deviation from the initial training output value across all ten patients is 0.037, while the average value of patient one only deviated by 0.012. This deviation, while lower than average, is not the lowest of all ten patients in trial 1; therefore, the lowest column displays N. Because the average deviation of patient one is greater than 0.01 the correct at 0.01 column also displays an N. However, the deviation is lower than 0.025 so the correct at 0.025 column (and the remaining correct at columns) display a Y value. In trial 1, no patients deviate from the initial output value by less than 0.01, as indicated in the False at 0.01 column. Five patients deviated by less than 0.025, seven patients deviated by less than 0.05, and eight patients deviated by less than 0.10.

TABLE 1
Set 1, trials 1-10 summary data (single 0.5 PR value).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.037 | 0.012 | N | N | Y | Y | Y | 0 | 5 | 7 | 8 |
| 0.044 | 0.035 | N | N | N | Y | Y | 1 | 5 | 7 | 8 |
| 0.129 | 0.064 | N | N | N | N | Y | 0 | 0 | 0 | 2 |
| 0.064 | 0.010 | N | N | Y | Y | Y | 1 | 2 | 6 | 6 |
| 0.034 | 0.033 | N | N | N | Y | Y | 3 | 5 | 7 | 8 |
| 0.045 | 0.004 | Y | Y | Y | Y | Y | 0 | 2 | 6 | 8 |
| 0.059 | 0.010 | N | N | Y | Y | Y | 1 | 3 | 4 | 8 |
| 0.056 | 0.024 | N | N | Y | Y | Y | 0 | 2 | 5 | 8 |
| 0.050 | 0.002 | Y | Y | Y | Y | Y | 2 | 3 | 5 | 8 |
| 0.153 | 0.100 | N | N | N | N | Y | 0 | 0 | 1 | 2 |

From these results, a ratio of correct-to-incorrect patients can be determined for each margin of error by examining the collection of all ten rows. In these ten trials, there were two correct patients that fell within a 0.01 margin of error (these being trials 6 and 9) while the trials averaged 0.8 incorrect patients also falling within this margin (with 0.8 being the average of the false at 0.01 column). This ratio is 2:0.8, or 2.5. At a 0.025 margin of error, this ratio is 2.22.

At a 0.05 error margin, the ratio is 1.66. Finally, at an error margin of 0.10, this ratio is 1.52. A high number of correct patients falling within a given error margin corresponding to a low number of incorrect patients also falling within that error margin means a high-performing model.

Each of the following tables highlight the highest and lowest performing collections of all trials. All remaining results are shown in Appendix B.

Table 2 displays the results from the lowest performing collection of trials that use the first GDES network model, with rows one through ten corresponding to trials 11 through 20, respectively. Because these are trials 11 through 20, the isolated data conversion method is used. The third PR approach is what was utilized for these ten trials. As seen in the table, the target patient often deviates from the initial training output value more than the average patient, indicating these trials do not favor the correct patient at any margin of error. This is the case in trials 3, 4, 6, 7, 8, and 9. No patients, correct or incorrect, fall within any listed margins of error.

TABLE 2
Set 1, trials 11-20 summary data (all 0.5 PR values).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.199 | 0.165 | N | N | N | N | N | 0 | 0 | 0 | 0 |
| 0.204 | 0.193 | N | N | N | N | N | 0 | 0 | 0 | 0 |
| 0.204 | 0.243 | N | N | N | N | N | 0 | 0 | 0 | 0 |
| 0.204 | 0.218 | N | N | N | N | N | 0 | 0 | 0 | 0 |
| 0.204 | 0.168 | N | N | N | N | N | 0 | 0 | 0 | 0 |
| 0.204 | 0.222 | N | N | N | N | N | 0 | 0 | 0 | 0 |
| 0.204 | 0.216 | N | N | N | N | N | 0 | 0 | 0 | 0 |
| 0.204 | 0.226 | N | N | N | N | N | 0 | 0 | 0 | 0 |
| 0.204 | 0.208 | N | N | N | N | N | 0 | 0 | 0 | 0 |
| 0.204 | 0.111 | Y | N | N | N | N | 0 | 0 | 0 | 0 |

Table 3 displays results from the highest performing collection of trials using the first GDES model. It is trials 21 through 30, indicating that the comprehensive data conversion method is used and that the default fact value is 000.600. In all but one trial (trial 8), the target patient deviates from the target value less than the average patient. The highest ratio of correct-to-incorrect patients is found at an error margin of 0.01. At this margin, three correct patients are included with an average of 0.6 incorrect also being included. Thus, the ratio is 5. Notably, at an error margin of 0.025, there are 6 correct patients included with an average of 2.1 incorrect patients per trial. While this ratio is lower, at 2.86, there are twice as many correct patients included within the margin. No other trial collections produced multiple ratios this high utilizing the first GDES network model, although other models do have higher-performing trials.

TABLE 3
Set 1, trials 21-30 summary data (actual PR value).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.053 | 0.024 | N | N | Y | Y | Y | 1 | 3 | 4 | 8 |
| 0.053 | 0.035 | N | N | N | Y | Y | 0 | 4 | 6 | 8 |
| 0.055 | 0.002 | Y | Y | Y | Y | Y | 0 | 1 | 4 | 8 |
| 0.076 | 0.016 | Y | N | Y | Y | Y | 0 | 2 | 3 | 7 |
| 0.049 | 0.030 | N | N | N | Y | Y | 0 | 2 | 7 | 8 |
| 0.061 | 0.002 | Y | Y | Y | Y | Y | 0 | 1 | 3 | 7 |
| 0.068 | 0.010 | N | N | Y | Y | Y | 1 | 3 | 3 | 7 |
| 0.062 | 0.085 | N | N | N | N | Y | 2 | 2 | 3 | 7 |
| 0.065 | 0.008 | N | Y | Y | Y | Y | 2 | 3 | 3 | 7 |
| 0.297 | 0.173 | N | N | N | N | N | 0 | 0 | 0 | 1 |

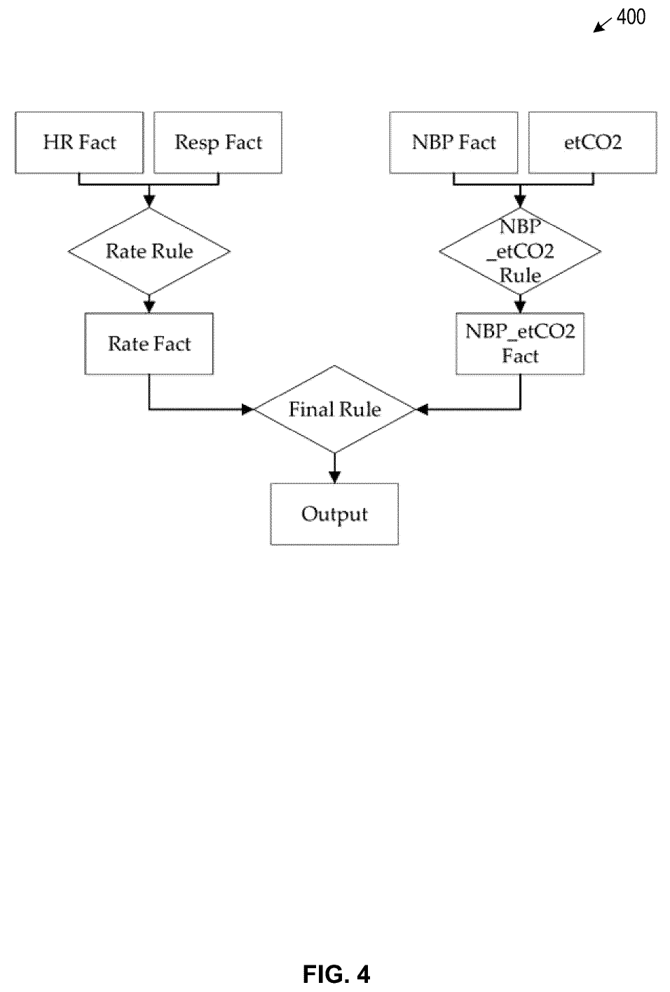

FIG. 4 is a diagram of a second GDES network 400, according to an embodiment.

The second network design, shown in FIG. 4, links heart rate and respiratory rate into a single rate rule while blood pressure and end-tidal carbon dioxide combine into another rule. These rules produce facts which serve as inputs to the final rule, which produces the output.

A similar set of tests were performed with this second network. Trials 1 through 10 utilized a default weight of 0.5 and the comprehensive data conversion method. Trials 11 through 20 used a default weight of 0.5 and the isolated data conversion method. Trials 21 through 50 utilized the comprehensive data conversion method with default weights of 0.6, 0.7, and 0.55 for trials 21 through 30, 31 through 40, and 41 through 50, respectively.

Table 4 displays the results of trials 1 through 10 utilizing the second GDES network model and the fourth PR approach, as described below. These trials are noteworthy as at an error margin of 0.01 there are four correct patients included with an average of 0.7 incorrect patients also included in the margin. This ratio, 4:0.7, or 5.71, is the second highest of any collection of trials. While this is noteworthy, the actual number of correct patients within the margin of error is only four, meaning that in most of the trials the correct patient did not fall within a 0.01 margin of error.

TABLE 4
Set 2, trials 1-10 summary data (average actual PR values).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.040 | 0.008 | N | Y | Y | Y | Y | 1 | 4 | 7 | 7 |
| 0.038 | 0.022 | N | N | Y | Y | Y | 2 | 5 | 7 | 8 |
| 0.161 | 0.037 | Y | N | N | Y | Y | 0 | 0 | 0 | 1 |
| 0.096 | 0.012 | Y | N | Y | Y | Y | 0 | 1 | 4 | 6 |
| 0.074 | 0.010 | Y | Y | Y | Y | Y | 0 | 0 | 4 | 7 |
| 0.069 | 0.036 | N | N | N | Y | Y | 0 | 3 | 3 | 7 |
| 0.053 | 0.061 | N | N | N | N | Y | 2 | 5 | 6 | 7 |
| 0.321 | 0.305 | N | N | N | N | N | 0 | 0 | 0 | 0 |
| 0.038 | 0.010 | N | Y | Y | Y | Y | 1 | 5 | 7 | 8 |
| 0.060 | 0.007 | N | Y | Y | Y | Y | 1 | 2 | 5 | 7 |

Table 5 represents the lowest performing collection of trials that utilize the second GDES network model. This is trials 11 through 20, indicating the use of the isolated data conversion method. The third PR approach is used. In trials 11, 13, 14, 17, 18, and 19 the target patient deviated from the target value more than the average of all patients. No patients, correct or incorrect, fall within any listed margin of error.

TABLE 5
Set 2, trials 11-20 data (all 0.5 PR values).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.402 | 0.407 | N | N | N | N | N | 0 | 0 | 0 | 0 |
| 0.423 | 0.384 | N | N | N | N | N | 0 | 0 | 0 | 0 |
| 0.423 | 0.457 | N | N | N | N | N | 0 | 0 | 0 | 0 |
| 0.423 | 0.468 | N | N | N | N | N | 0 | 0 | 0 | 0 |
| 0.423 | 0.418 | N | N | N | N | N | 0 | 0 | 0 | 0 |
| 0.423 | 0.334 | Y | N | N | N | N | 0 | 0 | 0 | 0 |
| 0.423 | 0.466 | N | N | N | N | N | 0 | 0 | 0 | 0 |
| 0.423 | 0.476 | N | N | N | N | N | 0 | 0 | 0 | 0 |
| 0.423 | 0.458 | N | N | N | N | N | 0 | 0 | 0 | 0 |
| 0.423 | 0.361 | N | N | N | N | N | 0 | 0 | 0 | 0 |

Table 6 displays the highest performing trial collection utilizing the second GDES network model. It represents trials 21 through 30, indicating the default fact value used is 000.600. The second PR approach is used. At a margin of error of 0.01 there are 3 correct patients included and an average of 0.6 incorrect per trial. This is a correct-to-incorrect ratio of 5. At a 0.025 margin of error 5 correct patients are included along with an average of 1.2 incorrect patients. This ratio is 4.17.

TABLE 6
Set 2, trials 21-30 summary data (actual PR value).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.050 | 0.006 | N | Y | Y | Y | Y | 2 | 3 | 4 | 8 |
| 0.072 | 0.055 | N | N | N | N | Y | 0 | 1 | 5 | 6 |
| 0.159 | 0.029 | Y | N | N | Y | Y | 0 | 0 | 0 | 1 |
| 0.055 | 0.011 | N | N | Y | Y | Y | 2 | 2 | 4 | 7 |
| 0.121 | 0.027 | Y | N | N | Y | Y | 0 | 0 | 1 | 3 |
| 0.053 | 0.009 | N | Y | Y | Y | Y | 1 | 1 | 4 | 8 |
| 0.071 | 0.040 | N | N | N | Y | Y | 0 | 1 | 3 | 7 |
| 0.206 | 0.115 | N | N | N | N | N | 0 | 0 | 0 | 0 |
| 0.042 | 0.002 | N | Y | Y | Y | Y | 1 | 4 | 6 | 8 |
| 0.124 | 0.021 | Y | N | Y | Y | Y | 0 | 0 | 0 | 2 |

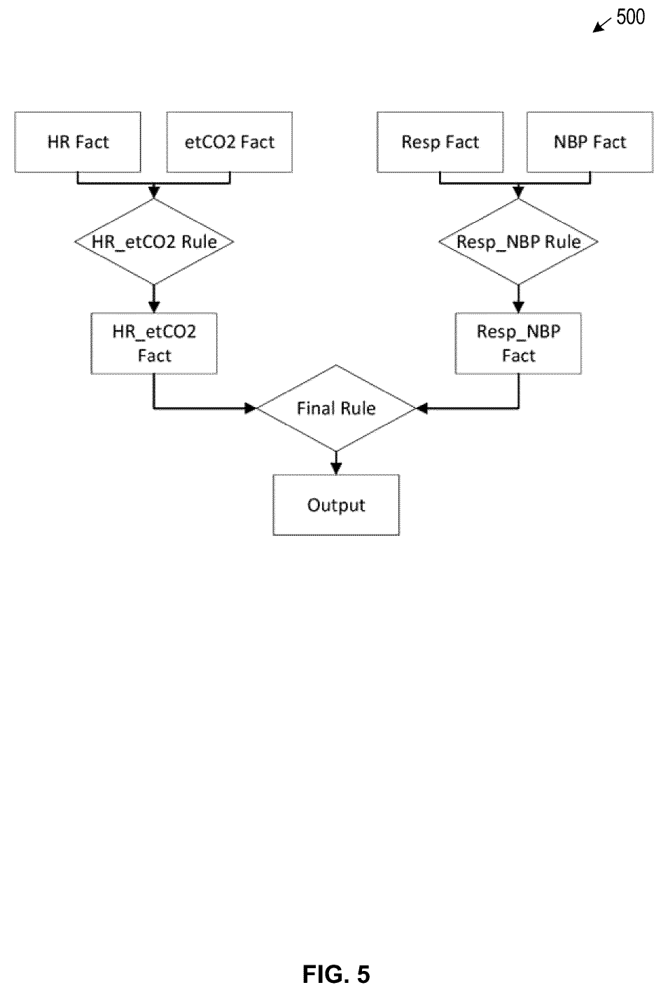

FIG. 5 is a diagram of a third GDES network 500, according to an embodiment.

The third network design, which is depicted in FIG. 5, combines the heart rate and end-tidal carbon dioxide facts with a single rule, while respiratory rate and blood pressure are combined using a second. These two rules produce facts that serve as inputs to the final rule, leading to the output fact. Notably, this network design tended to produce greater accuracy than the previous two networks, so results for this model will be explored in slightly more detail.

Table 7 is a noteworthy performer of the third GDES network model. It represents trials 1 through 10, so the default fact value used in this case is 000.500. It also uses the first PR approach described herein. As can be concluded from the average error and avg err columns, output from the target patient is always closer to the target value than the average patient. The most notable aspect of these trials is found at the 0.025 margin of error. Across seven of the trials, the correct patient falls within the margin. Meanwhile, across the trials, an average of 1.7 incorrect patients also fall within the margin. This is a 7:1.7 correct-to-incorrect ratio, or 4.12. The high ratio, along with seven correct patients falling within the margin of error, makes this trial collection one of the most accurate.

TABLE 7
Set 3, trials 1-10 summary data (single 0.5 PR value).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.056 | 0.005 | Y | Y | Y | Y | Y | 0 | 0 | 4 | 9 |
| 0.026 | 0.011 | N | N | Y | Y | Y | 3 | 4 | 7 | 9 |
| 0.133 | 0.096 | N | N | N | N | Y | 0 | 1 | 1 | 2 |
| 0.090 | 0.007 | Y | Y | Y | Y | Y | 1 | 2 | 4 | 6 |
| 0.028 | 0.000 | Y | Y | Y | Y | Y | 1 | 4 | 8 | 9 |
| 0.112 | 0.022 | Y | N | Y | Y | Y | 0 | 0 | 1 | 3 |
| 0.100 | 0.023 | Y | N | Y | Y | Y | 0 | 0 | 1 | 3 |
| 0.085 | 0.048 | N | N | N | Y | Y | 0 | 2 | 4 | 6 |
| 0.082 | 0.025 | N | N | Y | Y | Y | 1 | 2 | 4 | 6 |
| 0.069 | 0.052 | N | N | N | N | Y | 2 | 2 | 5 | 7 |

Table 8 represents an exceptional collection of trials for several reasons. It is trials 11 through 20, indicating the isolated data conversion method is used. It also utilizes the second PR approach. Specifically, these trials perform well at a 0.01 margin of error. While a relatively unimpressive four trials have included patients that fall within that margin, there is an average of only 0.5 incorrect patients also being included. This 4:0.5 ratio, or 8, is the highest of any collection of trials across the entirety of this experiment. Additionally, this is unexpected considering the isolated data conversion method generally underperforms. While this is a noteworthy ratio, the actual number of correct patients falling within the margin is not substantial in comparison to some of the other high performers, particularly those utilizing GDES model three.

TABLE 8
Set 3, trials 11-20 summary data (actual PR value).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.056 | 0.002 | Y | Y | Y | Y | Y | 2 | 4 | 6 | 8 |
| 0.054 | 0.009 | Y | Y | Y | Y | Y | 0 | 2 | 7 | 8 |
| 0.058 | 0.062 | N | N | N | N | Y | 1 | 2 | 7 | 8 |
| 0.092 | 0.007 | Y | Y | Y | Y | Y | 0 | 2 | 4 | 6 |
| 0.068 | 0.104 | N | N | N | N | N | 1 | 3 | 7 | 8 |
| 0.054 | 0.027 | N | N | N | Y | Y | 0 | 3 | 7 | 8 |
| 0.054 | 0.012 | N | N | Y | Y | Y | 0 | 2 | 7 | 8 |
| 0.084 | 0.047 | N | N | N | Y | Y | 0 | 1 | 4 | 7 |
| 0.049 | 0.004 | N | Y | Y | Y | Y | 1 | 5 | 7 | 8 |
| 0.093 | 0.198 | N | N | N | N | N | 0 | 1 | 1 | 6 |

Table 9 displays the lowest performing trials of set 3. In trials 13, 14, 16, 17, 18, and 19 the target patient deviated further from the target value than most other patients. It uses the isolated data conversion method and the third PR approach. Like the corresponding lowest performers of the other GDES models, no patients, correct or incorrect, fall within any listed margin of error. The commonalities between all of the lowest performers for each model are the isolated data conversion method and the third PR approach. This indicates that these tend to produce low-performing trials, especially when used in tandem.

### Table 9

Set 3, trials 11-20 summary data (all 0.5 PR values).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.396 | 0.357 | N | N | N | N | N | 0 | 0 | 0 | 0 |
| 0.416 | 0.415 | N | N | N | N | N | 0 | 0 | 0 | 0 |
| 0.416 | 0.477 | N | N | N | N | N | 0 | 0 | 0 | 0 |
| 0.416 | 0.464 | N | N | N | N | N | 0 | 0 | 0 | 0 |
| 0.416 | 0.380 | N | N | N | N | N | 0 | 0 | 0 | 0 |
| 0.416 | 0.452 | N | N | N | N | N | 0 | 0 | 0 | 0 |
| 0.416 | 0.437 | N | N | N | N | N | 0 | 0 | 0 | 0 |
| 0.416 | 0.453 | N | N | N | N | N | 0 | 0 | 0 | 0 |
| 0.416 | 0.443 | N | N | N | N | N | 0 | 0 | 0 | 0 |
| 0.416 | 0.164 | Y | N | N | N | N | 0 | 0 | 0 | 0 |

Table 10 displays the results of another high-performing trial collection of GDES model three. These are trials 21 through 30 and use the second PR approach. In all trials except 28, the target patient is closer to the target value than most other patients. At an error margin of 0.01, there are two correct patients that fall within the margin while an average of 0.4 incorrect patients are also included. This correct-to-incorrect patient ratio is then 5. Expanding to a 0.025 margin of error improves the results further. Five correct patients fall within the margin of error while an average of 0.9 incorrect patients are also included. This ratio is then 5.56. This ratio is the third highest correct-to-incorrect patient ratio across the entire experiment, and the highest ratio of any error margin that includes five or more incorrect patients.

TABLE 10
Set 3, trials 21-30 summary data (actual PR value).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.132 | 0.007 | Y | Y | Y | Y | Y | 0 | 0 | 0 | 2 |
| 0.075 | 0.041 | N | N | N | Y | Y | 0 | 0 | 4 | 7 |
| 0.082 | 0.029 | N | N | N | Y | Y | 0 | 1 | 1 | 7 |
| 0.071 | 0.015 | Y | N | Y | Y | Y | 0 | 0 | 3 | 6 |
| 0.082 | 0.057 | N | N | N | N | Y | 0 | 0 | 3 | 6 |
| 0.111 | 0.022 | Y | N | Y | Y | Y | 0 | 0 | 1 | 3 |
| 0.097 | 0.094 | N | N | N | N | Y | 1 | 1 | 1 | 5 |
| 0.061 | 0.086 | N | N | N | N | Y | 1 | 2 | 4 | 7 |
| 0.054 | 0.006 | N | Y | Y | Y | Y | 2 | 5 | 5 | 7 |
| 0.111 | 0.023 | Y | N | Y | Y | Y | 0 | 0 | 0 | 2 |

Table 11 displays the results of trials 41 through 50 of the third GDES network model and first PR approach. Of all trials, these ten produce the most desirable outcome. At a margin of error of 0.025 nine of ten correct patients are included with an average of 1.9 incorrect patients also falling within the margin. This ratio, 9:1.9 or 4.74 is among the very highest and the error margin includes substantially more correct patients than any other trial collection with a comparable ratio.

TABLE 11
Set 3, trials 41-50 summary data (single 0.5 PR value).

| | | | Correct | | | | False | | | |
|---|---|---|---|---|---|---|---|---|---|---|
| | | | At | At | At | At | At | At | At | At |
| Average Error | Avg Err | Lowest | 0.01 | 0.025 | 0.05 | 0.1 | 0.01 | 0.025 | 0.05 | 0.10 |
| 0.058 | 0.005 | Y | Y | Y | Y | Y | 0 | 0 | 4 | 8 |
| 0.029 | 0.011 | N | N | Y | Y | Y | 3 | 4 | 7 | 9 |
| 0.135 | 0.096 | N | N | N | N | Y | 0 | 1 | 1 | 2 |
| 0.075 | 0.010 | N | N | Y | Y | Y | 1 | 1 | 3 | 7 |
| 0.030 | 0.000 | Y | Y | Y | Y | Y | 1 | 3 | 8 | 9 |
| 0.112 | 0.022 | Y | N | Y | Y | Y | 0 | 0 | 1 | 3 |
| 0.099 | 0.023 | Y | N | Y | Y | Y | 0 | 0 | 1 | 3 |
| 0.048 | 0.018 | N | N | Y | Y | Y | 1 | 4 | 5 | 8 |
| 0.027 | 0.019 | N | N | Y | Y | Y | 2 | 6 | 7 | 9 |
| 0.070 | 0.013 | Y | N | Y | Y | Y | 0 | 0 | 2 | 8 |

Table 12 summarizes each of the high performers with a few key details in order to compare the variables present across trials. As can be seen, the third model tends to outperform the first and second. A default value of 000.600 tends to outperform the others, as well as using the actual PR value to determine the target value. Only error margins of 0.01 and 0.025 are present in the top performers. While some of these variables tend to outperform other trials on average, they are not strictly superior. For example, using the actual PR value tended to produce higher correct-to-incorrect patient ratios on average; however, using the single 0.5 PR value results in higher numbers of incorrect patients falling within a 0.025 margin of error. Ultimately, the last column in Table 12 displays the results of the top performer. That combination of model, default fact value, and PR approach demonstrated in this case the ability to eliminate 80% of incorrect patients on average per trial while only eliminating the target patient in one trial.

TABLE 12
Summary of standout data.

| Model (Set) | 1 | 2 | 3 | 3 | 3 | 3 |
|---|---|---|---|---|---|---|
| Default Fact | 000.600 | 000.600 | 000.500 | 000.500 | 000.600 | 000.550 |
| Value | | | | | | |
| PR Approach | Actual PR | Actual PR | Single 0.5 | Actual PR | Actual PR | Single 0.5 |
| | value | value | PR value | value | value | PR value |
| Best Error | 0.01 | 0.01 | 0.025 | 0.01 | 0.025 | 0.025 |
| Margin | | | | | | |
| Correct Patients | 3 | 3 | 7 | 4 | 5 | 9 |
| Avg Incorrect | 0.6 | 0.5 | 1.7 | 0.5 | 0.9 | 1.9 |
| Patients | | | | | | |
| Correct-to- | 5 | 5 | 4.12 | 8 | 5.56 | 4.74 |
| Incorrect Ratio | | | | | | |

This disclosure has presented and analyzed a prospective technology, which is designed to help medical providers recognize misidentified patients. Notably, this is not performed via specific patient identification but rather by providing support or refutation for presumed identifications. Thus, this system could be used to provide alerts indicating possible misidentification for human follow-up. It could also be potentially paired with other indicator subsystems as part of a multi-factor patient misidentification system.

To assess the efficacy of the proposed approach, several algorithms for identifying misidentified patients, using gradient descent expert systems, were evaluated in this work. These included three different network designs, multiple ways of preparing data to supply it to the network, and different ways of generating the target value for a given patient, based on their historic vital signs data.

There are several key outcomes from the analysis of the trial data presented herein. First, it was shown that the GDES network design itself clearly affects the efficacy of the system's ability to identify misidentified patients. Despite each of the three network designs utilizing the same starting fact values and the same number of facts and rules, there were clear differences in performance between the different network designs. In the case where the target value was generated with a single 0.5 input, the third network design outperformed the other two. This indicated a potential advantage associated with linking patient heart rates with end-tidal carbon dioxide and respiratory rates with blood pressure.

Second, it was shown that the comprehensive data conversion method consistently outperformed the isolated data conversion method. This is evident from the fact that trials 11 through 20 for each network design tended to be the least accurate, and these were the batches of trials that utilized the isolated conversion method.

The third area of analysis is the target value used for training. Of the values used, there was no clearly superior performer. Different networks performed better with different values.

Overall, the results obtained are quite promising. While none of the trials demonstrated the capability of positively identifying all patients by only their vital signs, many of the trials demonstrated a consistent ability to eliminate many—and in some cases—the majority of incorrect patients, allowing the system to provide an effective warning for many single patient mix-up scenarios. In particular, the best performers, listed in Table 12, show that the GDES system can effectively rule out incorrectly identified patients in the majority of cases.

There are several other factors, which were not explored in the present study, that are key topics for potential future work. One area is assessing the level of training data that is needed. In this regard, two key considerations exist. The first is to determine what level of training is most effective. To this end, future work can focus on assessing whether using all 60,000 data records, which were used for this study for training, provides the best results. The second area of consideration is to assess what the cost and benefit tradeoff of using less than the optimal amount of data is, as operating with lower amounts of data would allow the system to provide misidentification warnings with less input data and potentially learn about a patient more promptly, before a mix-up can occur.

Another area for potential future work is the development of additional networks and their assessment. These networks could use some or all of the input data used in this study and potentially augment it with additional data types. In particular, the data analyzed could be augmented with image data, which could potentially be collected using providers' tablet computers, providing another independent source of patient validation/misidentification warning.

In conjunction with the above, a third area of potential future work is the assessment of the efficacy of using other types of vital sign data. This analysis could compare the ease and cost of collection, the amount of data required and the performance of the system, presenting a trade-off analysis that could guide real-world implementation decision-making.

Overall, this work has shown the efficacy of using GDES in a different way from previous work, where network result values are compared to each other instead of presenting multiple subjects to a single network for classification. Additionally, this work has shown the potential promise of using patient vital sign data for misidentification prevention. The data and analysis presented have demonstrated a meaningful ability to eliminate incorrect patients using common vital sign data. Based on this initial work, a number of promising areas for additional work have been identified for future exploration.

The apparatuses and methods described above may include or be included in high-speed computers, communication and signal processing circuitry, single-processor module or multi-processor modules, single embedded processors or multiple embedded processors, multi-core processors, message information switches, and application-specific modules including multilayer or multi-chip modules. Such apparatuses may further be included as sub-components within a variety of other apparatuses (e.g., electronic systems), such as televisions, cellular telephones, personal computers (e.g., laptop computers, desktop computers, handheld computers, etc.), tablets (e.g., tablet computers), workstations, radios, video players, audio players (e.g., MP3 (Motion Picture Experts Group, Audio Layer 3) players), vehicles, medical devices (e.g., heart monitors, blood pressure monitors, etc.), set top boxes, and others.

In the detailed description and the claims, the term “on” used with respect to two or more elements (e.g., materials), one “on” the other, means at least some contact between the elements (e.g., between the materials). The term “over” means the elements (e.g., materials) are in close proximity, but possibly with one or more additional intervening elements (e.g., materials) such that contact is possible but not required. Neither “on” nor “over” implies any directionality as used herein unless stated as such.

In the detailed description and the claims, a list of items joined by the term “at least one of” may mean any combination of the listed items. For example, if items A and B are listed, then the phrase “at least one of A and B” means A only; B only; or A and B. In another example, if items A, B, and C are listed, then the phrase “at least one of A, B and C” means A only; B only; C only; A and B (excluding C); A and C (excluding B); B and C (excluding A); or all of A, B, and C. Item A may include a single element or multiple elements. Item B may include a single element or multiple elements. Item C may include a single element or multiple elements.

In the detailed description and the claims, a list of items joined by the term “one of” may mean only one of the list items. For example, if items A and B are listed, then the phrase “one of A and B” means A only (excluding B), or B only (excluding A). In another example, if items A, B, and C are listed, then the phrase “one of A, B and C” means A only; B only; or C only. Item A may include a single element or multiple elements. Item B may include a single element or multiple elements. Item C may include a single element or multiple elements.

The above description and the drawings illustrate some embodiments of the inventive subject matter to enable those skilled in the art to practice the embodiments of the inventive subject matter. Other embodiments may incorporate structural, logical, electrical, process, and other changes. Examples merely typify possible variations. Portions and features of some embodiments may be included in, or substituted for, those of others. Many other embodiments will be apparent to those of skill in the art upon reading and understanding the above description.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A system for human identification, the system comprising:
processing circuitry; and
one or more memory devices including instructions, which when executed by the processing circuitry, configure the processing circuitry to:
obtain biometric data related to a specific human to be identified;
store this biometric data over a time period;
identify patterns in this biometric data; and
compare newly presented biometric data to these patterns to perform identification.

2. The system of claim 1, where the system is used to identify a human patient in a hospital setting.

3. The system of claim 1, where the system is used to determine that a human patient has been incorrectly identified by an identification mechanism.

4. The system of claim 1, where the system is used to identify a user of a computing device.

5. The system of claim 1, where the system is used to identify a user of a mobile communications device.

6. The system of claim 1, where the system includes the use of a gradient descent expert system training mechanism.

7. The system of claim 6, further including:
a training mechanism;
a rule-fact network;
a training biometric data input mechanism;
a presentation biometric data input mechanism;
an identification processing mechanism;
an identification output mechanism;
the use of the training mechanism to update the rule-fact network based on data provided by the training biometric data input mechanism;
the use of the identification processing mechanism to make an identification determination based on the rule-fact network; and
providing identification information to a system user via the identification output mechanism.

8. A method for human identification, the method comprising:
obtaining biometric data related to a specific human to be identified;
storing this biometric data over a time period;
identifying patterns in this biometric data; and
comparing newly presented biometric data to these patterns to perform identification.

9. The method of claim 8, where the identification is made via a comparison of the patterns in the biometric data to the patterns of one or more patients.

10. The method of claim 9, where the identification includes the use of at least one of: similarity threshold, difference level comparison, and rule-fact network output comparison.

11. The method of claim 8, further including:
using a training mechanism to update a rule-fact network based on data provided by a training biometric data input mechanism;
using an identification processing mechanism to make an identification determination based on the rule-fact network; and
providing identification information to a system user via an identification output mechanism.

12. The method of claim 8, where the method is used to identify a human patient in a hospital setting.

13. The method of claim 8, where the method is used to determine that a human patient has been incorrectly identified by an identification mechanism.

14. The method of claim 8, where the method is used to identify a user of a computing device.

15. The method of claim 8, where the method is used to identify a user of a mobile communications device.

16. The method of claim 8, where the method includes the use of a gradient descent expert system training mechanism.

17. At least one machine-readable medium including instructions that, when executed by processing circuitry, cause the processing circuitry to perform operations comprising a plurality of the features described herein.

18. An apparatus comprising means for implementing a plurality of the features described herein.

19. The apparatus of claim 18, where the apparatus is used to determine that a human patient has been incorrectly identified by an identification mechanism.

20. The apparatus of claim 18, where the apparatus is used to identify a user of a mobile communications device.
